(19) United States
(12) Patent Application Publication
(10) Pub. No.: US 2026/0106032 A1
(43) Pub. Date: Apr. 16, 2026

(54) MEDICAL ASSISTANCE APPARATUS, MEDICAL ASSISTANCE SYSTEM, AND MEDICAL ASSISTANCE METHOD

(71) Applicant: OLYMPUS MEDICAL SYSTEMS CORP., Tokyo (JP)

(72) Inventor: Yusuke KATO, Saitama-shi (JP)

(73) Assignee: OLYMPUS MEDICAL SYSTEMS CORP., Tokyo (JP)

(21) Appl. No.: 19/421,595

(22) Filed: Dec. 16, 2025

Related U.S. Application Data
(63) Continuation of application No. PCT/JP2023/024276, filed on Jun. 29, 2023.

(51) Int. Cl. G16H 50/20; G16H 10/60; G16H 15/00; G16H 40/63; G16H 50/70; G16H 70/60

(52) U.S. Cl. CPC G16H 50/20; G16H 10/60; G16H 15/00; G16H 40/63; G16H 50/70; G16H 70/60

(57) ABSTRACT

A medical assistance apparatus including a processor including hardware. The processor being configured to: record an examination result for a patient; transmit a report based on the examination result to an external apparatus, infer, based on the report on the patient in a past, examination details and an examination timing of a subsequent examination of the patient; determine whether or not read-status information has been received from the external apparatus within a predetermined time period; and when it is determined that the read-status information has not been received within the predetermined time period, transmit a request for generation of an examination order according to the examination details and the examination timing of the subsequent examination of the patient to the external apparatus.

### CROSS REFERENCES TO RELATED APPLICATIONS

This application is a continuation of International Application No. PCT/JP2023/024276, filed on Jun. 29, 2023, the entire contents of which are incorporated herein by reference.

### BACKGROUND

### 1. Technical Field

The present disclosure relates to a medical assistance apparatus, a medical assistance system, and a medical assistance method that provide assistance in endoscopy.

### 2. Related Art

In a known technique for preventing medical reports from being overlooked in the medical field, a status is displayed on a display screen that displays, for each patient, identification information on the patient and a clinical process, such as a report, in association with each other, the status including a piece of information indicating that the clinical process has not been read yet, a piece of information indicating that the clinical process has been read, or both of these pieces of information (see, for example, Japanese Unexamined Patent Application, Publication No. 2020-004244). In this technique, in a case where a clinical process with a status marked as unread or read has been revised, the status of the clinical process that has been revised is displayed in a display mode different from display modes indicating that the clinical process has been read and has not been read yet, on the display screen that displays, for each patient, identification information on the patient and a clinical process in association with each other.

### SUMMARY

In some embodiments, a medical assistance apparatus includes a processor including hardware. The processor is configured to: record an examination result for a patient examined according to an examination request from an external apparatus interoperable with the medical assistance apparatus; transmit a report including an examination interval, qualitative diagnosis, and procedure information that are based on the examination result to the external apparatus, infer, based on the report on the patient in a past, examination details and an examination timing of a subsequent examination of the patient, by using an inference model that estimates the examination result from information on a record of endoscopic examinations and a pathology result; determine whether or not read-status information indicating that the examination result were read has been received from the external apparatus within a predetermined time period from transmission of the report to the external apparatus; and when it is determined that the read-status information has not been received within the predetermined time period, transmit a request for generation of an examination order according to the examination details and the examination timing of the subsequent examination of the patient to the external apparatus.

In some embodiments, a medical assistance system includes: a medical assistance apparatus comprising a processor comprising hardware; and an external apparatus interoperable with the medical assistance apparatus. The processor is configured to record an examination result for a patient examined according to an examination request from the external apparatus interoperable with the medical assistance apparatus, transmit a report based on the examination result to the external apparatus, infer, based on the examination result for the patient in a past, examination details and an examination timing of a subsequent examination of the patient, determine whether or not read-status information indicating that the examination result were read has been received from the external apparatus within a predetermined time period from transmission of the examination result to the external apparatus, and when it is determined that the read-status information has not been received within the predetermined time period, transmit a request for generation of an examination order according to the examination details and the examination timing of the subsequent examination of the patient to the external apparatus.

In some embodiments, provided is a medical assistance method executed by a medical assistance apparatus. The method includes: recording an examination result for a patient examined according to an examination request from the external apparatus interoperable with the medical assistance apparatus, transmitting a report based on the examination result to the external apparatus, inferring, based on the examination result for the patient in a past, examination details and an examination timing of a subsequent examination of the patient, determining whether or not read-status information indicating that the examination result were read has been received from the external apparatus within a predetermined time period from transmission of the examination result to the external apparatus, and when it is determined that the read-status information has not been received within the predetermined time period, transmitting a request for generation of an examination order according to the examination details and the examination timing of the subsequent examination of the patient to the external apparatus.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of a medical assistance system according to an embodiment;

FIG. 2 is a block diagram illustrating a functional configuration of a medical assistance apparatus according to the embodiment;

FIG. 3 is a sequence diagram illustrating an outline of a process executed by the medical assistance system according to the embodiment;

FIG. 4 is a diagram illustrating an example of a report prepared by a preparation unit included in the medical assistance apparatus according to the embodiment;

FIG. 5 is a diagram illustrating an example of a progression pattern of a case;

FIG. 6 is a diagram illustrating an example of an inference result inferred by a first inference unit included in the medical assistance apparatus according to the embodiment;

FIG. 7 is a diagram illustrating an example of input by an operating surgeon for acuity information on a patient according to pathology results; and

FIG. 8 is a diagram illustrating an example of an image displayed by an external apparatus according to the embodiment.

### DETAILED DESCRIPTION

An embodiment according to the present disclosure will hereinafter be described in detail with reference to the drawings. The present disclosure is not to be limited by the following embodiment. The drawings referred to in the following description merely illustrate shapes, sizes, and positional relations schematically to enable the present disclosure to be understood. That is, the present disclosure is not to be limited only to the shapes, sizes, and positional relations exemplified by the drawings. Furthermore, like reference signs will be assigned to like parts in the drawings.

### Overall Configuration of Medical Assistance System

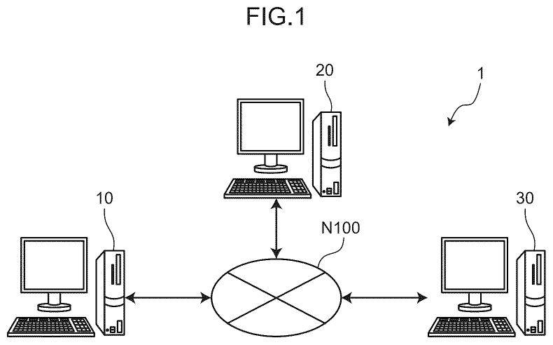

FIG. 1 is a diagram illustrating an overall configuration of a medical assistance system according to an embodiment. A medical assistance system 1 illustrated in FIG. 1 includes: a medical assistance apparatus 10 that functions as an endoscope management apparatus or an endoscope management system that manages image data groups captured by endoscopes and results of endoscopic examinations of patients; an external apparatus 20 that functions as an electronic medical record apparatus or an electronic medical record system that manages medical information on and the date of birth of each patient; and a pathology management apparatus 30 that functions as a pathology management apparatus or a pathology management system that manages pathology results of pathological examinations of specimens obtained by endoscopic biopsy, for example.

The medical assistance apparatus 10, the external apparatus 20, and the pathology management apparatus 30 are connected bidirectionally to each other via a network N100. The network N100 includes, for example, the Internet communication network or a mobile communication network.

### Functional Configuration of Medical Assistance Apparatus

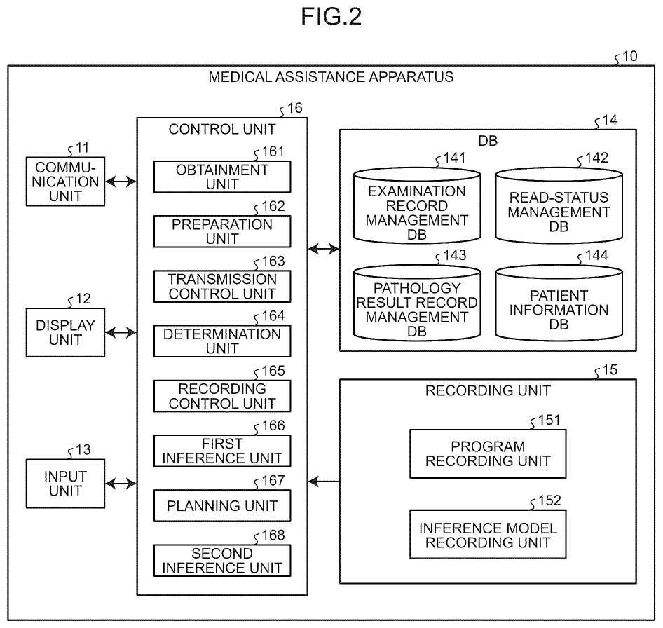

A functional configuration of the medical assistance apparatus 10 will be described next. FIG. 2 is a block diagram illustrating the functional configuration of the medical assistance apparatus 10. The medical assistance apparatus 10 illustrated in FIG. 2 includes a communication unit 11, a display unit 12, an input unit 13, a database 14 (hereinafter, simply referred to as “DB 14”), a recording unit 15, and a control unit 16.

Under control by the control unit 16, the communication unit 11 transmits various information to the external apparatus 20 and the pathology management apparatus 30 and receives various information from the external apparatus 20 and the pathology management apparatus 30, via the network N100. The communication unit 11 is configured using a communication module.

Under control by the control unit 16, the display unit 12 displays various information related to the medical assistance apparatus 10. The display unit 12 is configured using, for example, a liquid crystal display or an organic electroluminescent (EL) display.

According to operations from the outside, the input unit 13 receives various input and outputs the various input received, to the control unit 16. The input unit 13 is configured using any of buttons, a keyboard, switches, and a touch panel, for example.

The DB 14 has, recorded therein, various information. The DB 14 is configured using an HDD and an SSD, for example. The DB 14 has an examination record management DB 141, a read-status management DB 142, a pathology result record management DB 143, and a patient information DB 144.

The examination record management DB 141 has, recorded therein, examination record information related to results of an examination with an endoscope for each patient and a record of examinations with an endoscope for each patient. Specifically, the examination record management DB 141 has, recorded therein, an examination date, an examination type, an examination interval, qualitative diagnosis, procedure information indicating procedure details, an examination report, and image data, in association with each patient ID identifying a patient.

The read-status management DB 142 has, recorded therein, read-status information for each examination ID (each examination number) or each report based on examination results, included in the examination record information recorded in the examination record management DB 141. The read-status information is information or a flag indicating that a medical doctor from another department has read the examination results or the report on the examination results by checking the examination results or report and pressing a “Read” button, for example, using the external apparatus 20.

The pathology result record management DB 143 has, recorded therein, pathological examination results of a pathological examination of a specimen obtained by endoscopic biopsy or biopsy using a treatment tool, for each patient ID and each examination ID.

The patient information DB 144 has, recorded therein, for each patient, a patient ID identifying the patient, the patient's medical visit history, the patient's date of birth, a medical doctor in charge of the patient, the patient's address, and allergy information on the patient.

The recording unit 15 is configured using a volatile memory, a non-volatile memory, an HDD, and an SSD, for example. The recording unit 15 has a program recording unit 151 and an inference model recording unit 152.

The program recording unit 151 has, recorded therein, various programs and parameters for operating the medical assistance apparatus 10.

The inference model recording unit 152 has, recorded therein, an inference model that is used to estimate an examination result. The inference model has, as input, information on records of endoscopic examinations and pathology results, and as output, acuity information on acuity of patients. Acuity herein refers to a patient's stage or lesion level, for example. The inference model is, for example, a model generated by machine learning using artificial intelligence (AI). Specifically, the inference model is a deep learning model that is trained using training data having labels (points) assigned to examination record information and pathology result information and that outputs a stage or a lesion level of a patient for examination record information on the patient.

The control unit 16 corresponds to a processor according to the present disclosure. The control unit 16 is implemented using: a processor having hardware, such as a field-programmable gate array (FPGA), a graphics processing unit (GPU), or a central processing unit (CPU); and a memory that is a temporary storage area used by the processor. The control unit 16 integrally controls each unit included in the medical assistance apparatus 10. The control unit 16 has an obtainment unit 161, a preparation unit 162, a transmission control unit 163, a determination unit 164, a recording control unit 165, a first inference unit 166, a planning unit 167, and a second inference unit 168.

The obtainment unit 161 obtains read-status information from the external apparatus 20 via the network N100 and the communication unit 11. Furthermore, the obtainment unit 161 obtains pathology results from the pathology management apparatus 30 via the network N100 and the communication unit 11.

On the basis of an operation on the input unit 13 by an operating surgeon, such as a medical doctor in an endoscopy department, the preparation unit 162 prepares a report (examination report) based on examination results for a patient, for each patient ID.

The transmission control unit 163 transmits a report preparation completion notification indicating that preparation of the report prepared by the preparation unit 162 has been completed and the report, to the external apparatus 20.

The determination unit 164 determines whether or not read-status information on the report has been received from the external apparatus 20 within a predetermined time period, for example, one week, after the transmission of the report preparation completion notification by the transmission control unit 163 to the external apparatus 20 or within a predetermined time period, for example, one week, after the transmission of the report from the transmission control unit 163 to the external apparatus 20.

The recording control unit 165 records any one or more of a patient ID, a report ID, and an examination ID, in association with read-status information, into the read-status management DB 142.

On the basis of examination records recorded in the examination record management DB 141, the inference model recorded in the inference model recording unit 152, and a history of examination results for a patient corresponding to a report that has not been read yet, the first inference unit 166 infers, for the unread report on an examination of the patient, a subsequent examination subsequent to that examination.

On the basis of necessity of an examination inferred by the second inference unit 168, the planning unit 167 formulates a treatment plan for a relevant patient.

On the basis of statistical information based on pathology results and examination results for other patients, the second inference unit 168 infers necessity of a subsequent examination.

### Process in Medical Assistance System

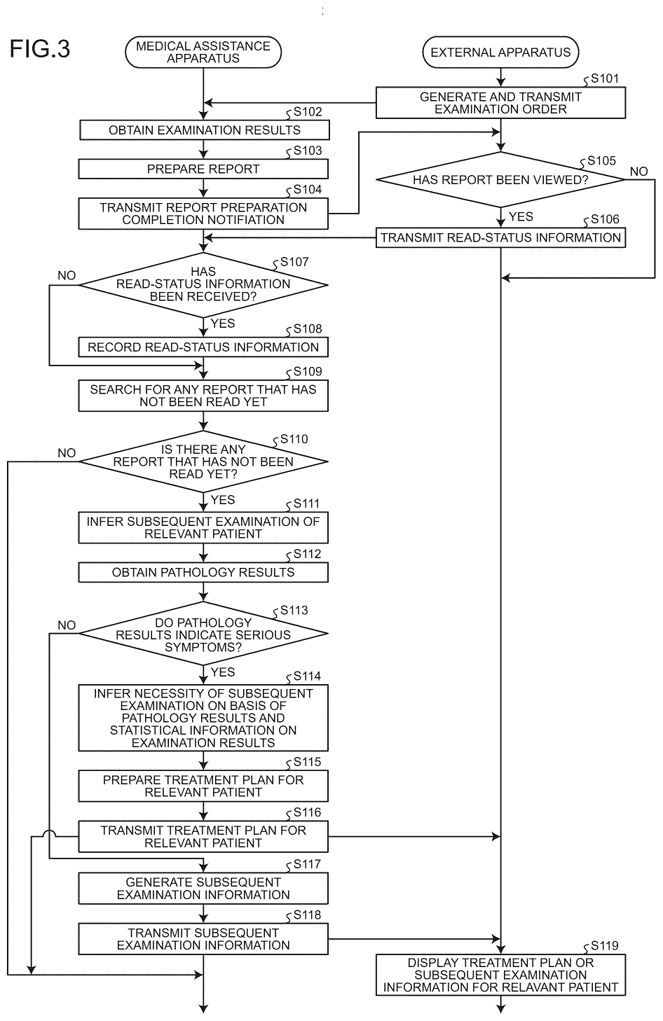

A process executed by the medical assistance system 1 will be described next. FIG. 3 is a sequence diagram illustrating an outline of the process executed by the medical assistance system 1.

As illustrated in FIG. 3, on the basis of an operation by a medical doctor from another department on an input device, such as a mouse or a keyboard, the external apparatus 20 generates an examination order (examination request) for an endoscopic examination of a patient and transmits the examination order to the medical assistance apparatus 10 (Step S101). In this case, the external apparatus 20 receives an operation based on clinical findings by the medical doctor from that other department via the input device, generates the examination order including a patient ID, an examination date, an examination type, and procedure details, according to the operation received, and transmits the examination order to the medical assistance apparatus 10. According to the examination order received at the medical assistance apparatus 10, an operating surgeon, such as a medical doctor in the endoscopy department, performs the endoscopic examination of the patient using an endoscope.

Subsequently, the obtainment unit 161 of the medical assistance apparatus 10 obtains examination results for each patient from an endoscope and records the examination results into the examination record management DB 141 (Step S102). In this case, the obtainment unit 161 records an examination ID identifying an examination order and the examination results from the endoscope, in association with a patient ID, into the examination record management DB 141. The examination results from the endoscope include an image data group and a still image captured by the endoscope, the time and date at which the still image was captured, and image recognition results including a feature area, such as a lesion area including cancer or a tumor, the image recognition results being from an image diagnosis apparatus (computer aided detection: CAD).

Thereafter, on the basis of an operation on the input unit 13 by an operating surgeon, such as a medical doctor in the endoscopy department, the planning unit 167 prepares a report (examination report) based on examination results for a patient, for each patient ID (Step S103). In this case, the planning unit 167 records the prepared report in association with the patient ID, into the examination record management DB 141.

Subsequently, the transmission control unit 163 transmits a report preparation completion notification indicating that report preparation has been completed, to the external apparatus 20. On the basis of a notification displayed on the external apparatus 20, a medical doctor from another department is thereby able to know that an endoscopic examination in the endoscopy department has been completed and a report has become viewable.

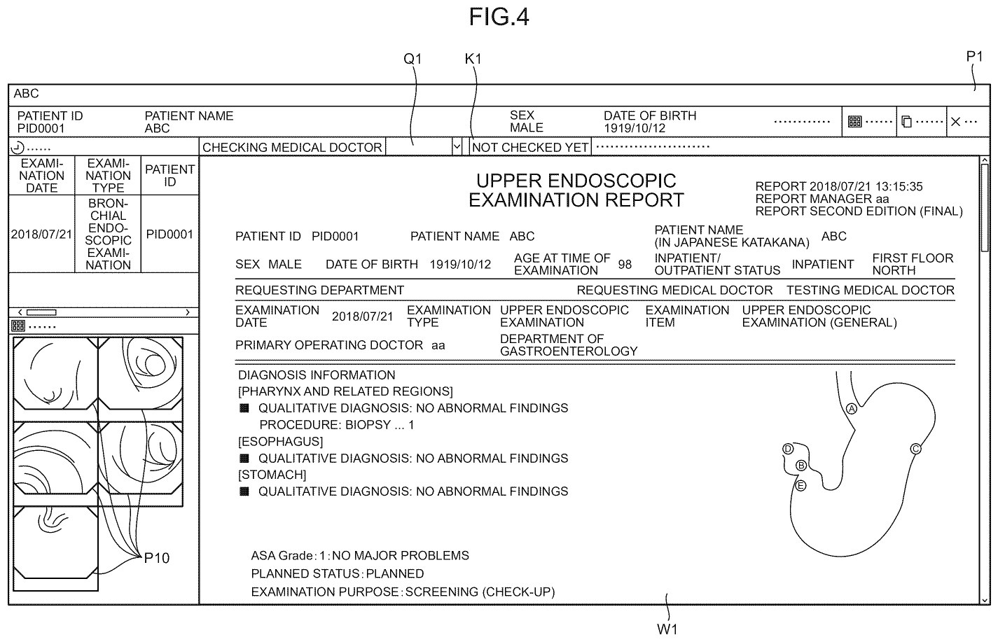

FIG. 4 is a diagram illustrating an example of a report prepared by the planning unit 167. A report P1 illustrated in FIG. 4 includes at least: an examination report W1 from image interpretation based on an image data group captured by an operating surgeon using an endoscope; more than one still image P10; an examination date; an examination type; a patient ID; a patient's full name; the patient's sex; the patient's date of birth; a checking medical doctor Q1; and a status K1 indicating a viewing status of the report P1. A medical doctor from another department operates an input device of the external apparatus 20, and in a case where the medical doctor has checked the report P1, the medical doctor enters the medical doctor's name and the medical doctor's medical doctor ID in the checking medical doctor Q1 field, and transitions the status K1 from an unchecked state to a checked state.

By reference back to FIG. 3, description of the process from Step S105 will be continued.

At Step S105, on the basis of an operation on the input device performed by a medical doctor, the external apparatus 20 determines whether or not the report has been viewed, according to the report preparation completion notification transmitted from the medical assistance apparatus 10 (Step S105). Specifically, the external apparatus 20 determines that a medical doctor from another department has viewed the report transmitted from the medical assistance apparatus 10, in a case where the medical doctor from the other department has accessed the medical assistance apparatus 10 by operating the input device of the external apparatus 20, has viewed, via the medical assistance apparatus 10, the report corresponding to the report preparation completion notification, and has transitioned the status K1 from the unchecked state to the checked state. In a case where the external apparatus 20 determines that the medical doctor from the other department has viewed the report according to the report preparation completion notification transmitted from the medical assistance apparatus 10 (Step S105: Yes), the external apparatus 20 transmits read-status information indicating that the report has been read by being viewed by the medical doctor, to the medical assistance apparatus 10 (Step S106). After step S106, the external apparatus 20 proceeds to Step S117 described later. In this case, the medical doctor from the other department prepares a treatment plan for the patient according to the content of the report. On the contrary, in a case where the external apparatus 20 determines that the medical doctor from the other department has not viewed the report according to the report preparation completion notification transmitted from the medical assistance apparatus 10 (Step S105: Yes), the external apparatus 20 proceeds to Step S119 described later.

At Step S107, the determination unit 164 determines whether or not read-status information on the report has been received from the external apparatus 20 within a predetermined time period, for example, within one week, from the transmission of the report preparation completion notification by the transmission control unit 163. In a case where the determination unit 164 determines that the read-status information on the report has been received from the external apparatus 20 within the predetermined time period from the transmission of the report preparation completion notification by the transmission control unit 163 (Step S107: Yes), the medical assistance apparatus 10 proceeds to Step S109 described later. On the contrary, in a case where the determination unit 164 determines that the read-status information on the report has not been received from the external apparatus 20 within the predetermined time period from the transmission of the report preparation completion notification by the transmission control unit 163 (Step S107: No), the medical assistance apparatus 10 proceeds to Step S109 described later.

At Step S108, the recording control unit 165 records any one or more of the patient ID, the report ID, and the examination ID, in association with the read-status information, into the read-status management DB 142.

Subsequently, on the basis of read-status information on each report recorded by the read-status management DB 142, the determination unit 164 searches for any report that has not been read yet (Step S109) and determines whether or not there is any report that has not been read yet in the read-status management DB 142 (Step S110). In a case where the determination unit 164 determines that there is any report that has not been read yet in the read-status management DB 142 (Step S110: Yes), the medical assistance apparatus 10 proceeds to Step S111 described later. On the contrary, in a case where the determination unit 164 determines that there is not any report that has not been read yet in the read-status management DB 142 (Step S110: No), the medical assistance apparatus 10 ends the process.

At Step S111, on the basis of examination records recorded in the examination record management DB 141, the inference model recorded in the inference model recording unit 152, and a history of results of examinations for a patient corresponding to a report that has not been read yet, the first inference unit 166 infers, for the unread report on results of an examination of the patient, a subsequent examination subsequent to that examination.

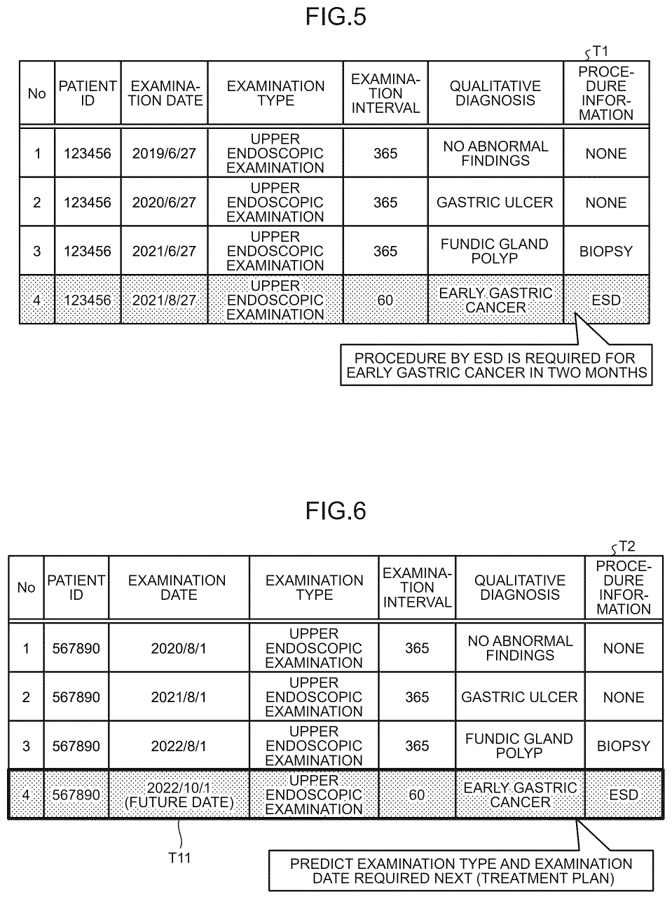

FIG. 5 is a diagram illustrating an example of a progression pattern of a case. FIG. 6 is a diagram illustrating an example of an inference result inferred by the first inference unit 166. A progression pattern T1 illustrated in FIG. 5 represents a case in which an examination was performed every year, and two months after detection of a fundic gland polyp, endoscopic submucosal dissection (ESD) was required for early gastric cancer.

As illustrated in FIG. 6, the first inference unit 166 infers an examination time and date T11 including a subsequent examination type, an examination interval, qualitative diagnosis, and procedure information, for a relevant patient corresponding to an unread report on examination results, on the basis of: examination records in the examination record management DB 141 having, recorded therein, more than one progression pattern T1 like the one in FIG. 5; the inference model recorded in the inference model recording unit 152; and a history T2 of detection results for the patient corresponding to the unread report. That is, the first inference unit 166 infers a treatment plan for the patient corresponding to the unread report on examination results.

By reference back to FIG. 3, description of the process from Step S112 will be continued.

At Step S112, the obtainment unit 161 obtains pathology results from the pathology management apparatus 30 via the network N100 and the communication unit 11. In this case, an operating surgeon, such as a medical doctor in the endoscopic department, enters acuity information related to acuity based on the pathology results.

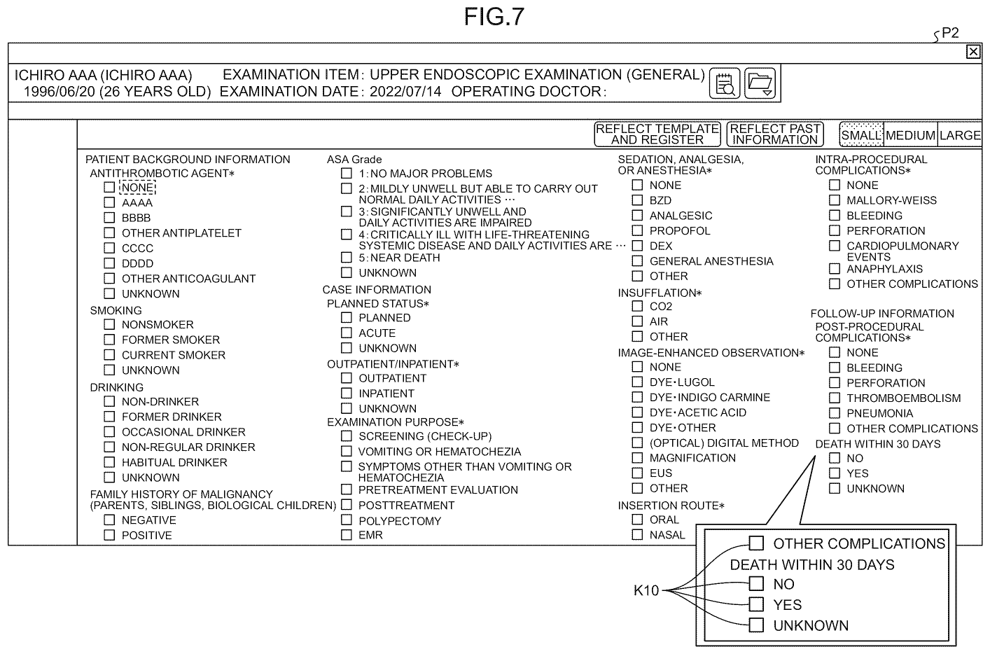

FIG. 7 is a diagram illustrating an example of input by an operating surgeon for acuity information on a patient according to pathology results. The operating surgeon operates the input unit 13 to enter the acuity information based on the pathology results, on an examination information screen P2 illustrated in FIG. 7. For example, in the case illustrated in FIG. 7, the operating surgeon selects “Yes” from multiple items K10 on the basis of the pathology results by operating the input unit 13.

Thereafter, on the basis of the pathology results obtained by the obtainment unit 161, the determination unit 164 determines whether or not the pathology results for the patient whose report has not been read yet indicate serious symptoms (Step S113). Specifically, on the basis of the acuity information related to the acuity corresponding to the item selected by the operating surgeon on the basis of the pathology results obtained by the obtainment unit 161, the determination unit 164 determines whether or not the pathology results for the patient indicate serious symptoms by determining whether or not the acuity is at a predetermined threshold or more. For example, in case where the item, “Yes” or “Unknown”, has been selected by the operating surgeon for “Death within 30 days” on the basis of the pathology results obtained by the obtainment unit 161, the determination unit 164 determines that the acuity is at the predetermined threshold or more and determines that the pathology results for the patient whose report has not been read yet indicate serious symptoms.

FIG. 7 described above illustrate three stages, “No”, “Yes”, and “Unknown”, for the items K10 related to death but without being limited to this example, the items K10 may have numerical values, or three stages, “Small”, “Medium”, and “Large”, for example.

In a case where the determination unit 164 determines that the pathology results for the patient whose report has not been read yet indicate serious symptoms (Step S113: Yes), the medical assistance apparatus 10 proceeds to Step S114 described later. On the contrary, in a case where the determination unit 164 determines that the pathology results for the patient whose report has not been read yet do not indicate serious symptoms (Step S113: No), the medical assistance apparatus 10 proceeds to Step S118 described later.

At Step S114, the second inference unit 168 infers necessity of a subsequent examination on the basis of the acuity information based on the pathology results, statistical information based on respective examination results of other patients, and past examination results for the patient. Specifically, the second inference unit 168 infers examination details and the examination timing of any subsequent examination, according to, in addition to an inference method that is the same as that described with respect to FIG. 6, the acuity information. In this case, additionally to the inference method that is the same as that described with respect to FIG. 6, when the acuity information indicates high acuity, for example, when the item for “Death within 30 days”, is “Yes”, the second inference unit 168 infers examination details and the examination timing of any subsequent examination for the patient so that the examination timing will be earlier than a conventional time and date. More specifically, if the examination time and date has been set to a date 60 days later (the examination interval is 60 days) according to the inference method described above with respect to FIG. 6, the second inference unit 168 infers examination details and the examination timing for any subsequent examination of the patient so that the examination time and date will be within 30 days. The second inference unit 168 may infer examination details and the examination timing of any subsequent examination for the patient by referring to, for example, Japanese Patent No. 4879368.

On the basis of necessity of the examination inferred by the second inference unit 168, the planning unit 167 prepares a treatment plan for the relevant patient (Step S115). Specifically, on the basis of the examination details, examination timing, and appointment information on an endoscopic examination, in inference results inferred by the second inference unit 168, the planning unit 167 formulates a treatment plan for the relevant patient for which a time and date has been reserved from available examination times and dates beforehand.

Subsequently, the transmission control unit 163 transmits a treatment plan for the relevant patient formulated by the planning unit 167, to the external apparatus 20 (Step S116). After step S116, the medical assistance apparatus 10 ends the process.

At Step S117, on the basis of inference results inferred by the first inference unit 166, the planning unit 167 generates subsequent examination information inferred with respect to the relevant patient.

Subsequently, the transmission control unit 163 transmits the subsequent examination information inferred with respect to the relevant patient and generated by the planning unit 167, to the external apparatus 20 (Step S118). After Step S118, the external apparatus 20 ends the process.

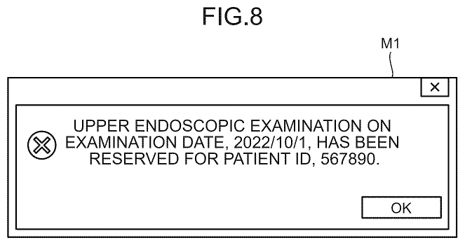

At Step S119, the external apparatus 20 displays the treatment plan or subsequent examination information for the relevant patient, transmitted from the medical assistance apparatus 10. Specifically, as illustrated in FIG. 8, the external apparatus 20 displays a treatment plan M1. A medical doctor is thereby able to know that an examination order for a scheduled examination date and an examination type has been made for the relevant patient. In this case, a medical doctor in charge is able to contact the relevant patient with high acuity. After Step S119, the external apparatus 20 ends the process. In a case where the external apparatus 20 has received subsequent examination information from the medical assistance apparatus 10, the external apparatus 20 displays the subsequent examination information. The medical doctor is thereby able to know that a report on the relevant patient has not been read yet.

According to the embodiment described above, in a case where the determination unit 164 determines that read-status information has not been received from the external apparatus 20 within a predetermined time period after transmission of a report preparation completion notification to the external apparatus 20, the transmission control unit 163 transmits a request for generation of an examination order according to examination details and the examination timing of any subsequent examination for the patient, to the external apparatus 20, and assistance is thus able to be provided in formulation of an appropriate treatment plan.

Furthermore, according to the embodiment, on the basis of acuity information based on pathology results, statistical information based on respective examination results of other patients, and past examination results for the patient, the second inference unit 168 infers examination details and the examination timing for any subsequent examination, and a medical doctor is thus able to be prompted to view a report on the patient requiring urgent attention.

Furthermore, according to the embodiment, the planning unit 167 reserves the examination details and an examination time and date according to the request for generation of the examination order, the transmission control unit 163 transmits, as a request for generation of an examination order, information indicating that the planning unit 167 has reserved the examination details and examination time and date according to the request for the generation of the examination order, to the external apparatus 20, and a medical doctor from another department is thus able to be prompted to prioritize viewing for the patient requiring urgent attention and an appointment for the examination order is thus able to be made automatically.

Furthermore, according to the embodiment, in a case where the determination unit 164 determines that acuity is not at a predetermined threshold or more, the transmission control unit 163 transmits a request for generation of an examination order according to examination details and the examination timing of any subsequent examination of the patient, to the external apparatus 20, and a medical doctor from another department is thus able to be prompted to view a report that has not been read yet and prompted to perform an appropriate examination.

Furthermore, according to the embodiment, the first inference unit 166 infers the examination details and the examination timing of any subsequent examination of the patient on the basis of the statistical information based on the respective examination results for multiple others, and precise examination details and examination timing are thus able to be submitted to a medical doctor from another department.

According to the embodiment, the first inference unit 166 infers the examination details and examination timing of any subsequent examination of the patient on the basis of the statistical information based on the respective examination results and examination records of multiple others, but without being limited to this example, multiple sets of statistical information based on respective examination results and examination records of multiple others respectively obtained by other multiple institutions may be obtained and examination details and the examination timing of any subsequent examination of the patient may be inferred on the basis of these multiple sets of statistical information. A more precise and appropriate examination is thereby able to be prompted.

Various embodiments may be formed by combination, as appropriate, of more than one component disclosed with respect to the above described medical assistance system according to the embodiment of the present disclosure. For example, some of all of the components described with respect to the medical assistance system according to the above described embodiment of the present disclosure may be eliminated. Furthermore, any components described with respect to the medical assistance system according to the above described embodiment of the present disclosure may be combined as appropriate.

Furthermore, any “unit” described above with respect to the medical assistance system according to the embodiment of the present disclosure may be read, for example, as a “means” or a “circuit”. For example, a control unit may be read as a control means or a control circuit.

Furthermore, programs to be executed by the medical assistance system according to the embodiment of the present disclosure are provided by being recorded as file data in an installable format or executable format in a computer-readable recording medium, such as a CD-ROM, a flexible disk (FD), a CD-R, a digital versatile disk (DVD), a USB medium, or a flash memory.

Furthermore, the programs to be executed by the medical assistance system according to the embodiment of the present disclosure may be configured to be stored on a computer connected to a network, such as the Internet, and to be provided by being downloaded via the network.

In the description of the flowcharts in this specification, the order of the steps in each process is disclosed by use of expressions, such as “firstly”, “thereafter”, and “subsequently”, but the order in each process needed for implementation of the disclosure is not uniquely defined by these expressions. That is, the order in the process in each flowchart described in this specification may be modified so long as no contradiction arises from that modification. Without being limited to such programs consisting of simple branching processes, a larger number of decision items may be comprehensively determined and branched.

According to the present disclosure, an effect of enabling assistance to be provided in formulation of appropriate treatment plans is provided.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical assistance apparatus comprising a processor comprising hardware, the processor being configured to:
record an examination result for a patient examined according to an examination request from an external apparatus interoperable with the medical assistance apparatus;
transmit a report including an examination interval, qualitative diagnosis, and procedure information that are based on the examination result to the external apparatus,
infer, based on the report on the patient in a past, examination details and an examination timing of a subsequent examination of the patient, by using an inference model that estimates the examination result from information on a record of endoscopic examinations and a pathology result;
determine whether or not read-status information indicating that the examination result were read has been received from the external apparatus within a predetermined time period from transmission of the report to the external apparatus; and
when it is determined that the read-status information has not been received within the predetermined time period, transmit a request for generation of an examination order according to the examination details and the examination timing of the subsequent examination of the patient to the external apparatus.

2. The medical assistance apparatus according to claim 1, wherein the processor is configured to
obtain a pathology result of a pathological examination of the patient,
estimate acuity information related to acuity based on the obtained pathology result, and
infer the examination details and the examination timing of the subsequent examination of the patient based on an examination result for the patient in the past and the acuity information.

3. The medical assistance apparatus according to claim 2, wherein the processor is configured to
determine, based on the acuity information, whether or not the acuity is at a predetermined threshold or more,
when it is determined that the acuity is at the predetermined threshold or more, reserve examination details and an examination time and date according to the request for the generation of the examination order, and
transmit, to the external apparatus, information indicating that the examination details and the examination time and date according to the request for the generation of the examination order have been reserved, as the request for the generation of the examination order.

4. The medical assistance apparatus according to claim 3, wherein when it is determined that the acuity is not at the predetermined threshold or more, the processor is configured to transmit, to the external apparatus, the request for the generation of the examination order according to the examination details and the examination timing of the subsequent examination of the patient.

5. The medical assistance apparatus according to claim 4, wherein the processor is configured to infer the examination details and the examination timing of the subsequent examination of the patient based on statistical information based on respective examination results of other patients.

6. The medical assistance apparatus according to claim 5, wherein
the medical assistance apparatus is an endoscope management apparatus configured to manage an endoscopic examination result, and
the external apparatus is an electronic medical record apparatus configured to manage information related to the patient.

7. A medical assistance system, comprising:
a medical assistance apparatus comprising a processor comprising hardware; and
an external apparatus interoperable with the medical assistance apparatus, wherein
the processor is configured to
record an examination result for a patient examined according to an examination request from the external apparatus interoperable with the medical assistance apparatus,
transmit a report based on the examination result to the external apparatus,
infer, based on the examination result for the patient in a past, examination details and an examination timing of a subsequent examination of the patient,
determine whether or not read-status information indicating that the examination result were read has been received from the external apparatus within a predetermined time period from transmission of the examination result to the external apparatus, and
when it is determined that the read-status information has not been received within the predetermined time period, transmit a request for generation of an examination order according to the examination details and the examination timing of the subsequent examination of the patient to the external apparatus.

8. The medical assistance system according to claim 7, wherein the processor is configured to
obtain a pathology result of a pathological examination of the patient,
estimate acuity information related to acuity based on the pathology result, and
infer the examination details and the examination timing of the subsequent examination of the patient based on the examination result for the patient in the past and the acuity information.

9. The medical assistance system according to claim 8, wherein the processor is configured to
determine, based on the acuity information, whether or not the acuity is at a predetermined threshold or more,
when it is determined that the acuity is at the predetermined threshold or more, reserve examination details and an examination time and date according to the request for the generation of the examination order, and
transmit, to the external apparatus, information indicating that the examination details and the examination time and date according to the request for the generation of the examination order have been reserved, as the request for the generation of the examination order.

10. The medical assistance system according to claim 9, wherein when it is determined that the acuity is not at the predetermined threshold or more, the processor is configured to transmit, to the external apparatus, the request for the generation of the examination order according to the examination details and the examination timing of the subsequent examination of the patient.

11. The medical assistance system according to claim 8, wherein the processor is configured to infer the examination details and the examination timing of the subsequent examination of the patient based on statistical information based on respective examination results of other patients.

12. The medical assistance system according to claim 9, wherein
the medical assistance apparatus is an endoscope management apparatus configured to manage an endoscopic examination result, and
the external apparatus is an electronic medical record apparatus configured to manage information related to the patient.

13. A medical assistance method executed by a medical assistance apparatus, the method comprising:
recording an examination result for a patient examined according to an examination request from the external apparatus interoperable with the medical assistance apparatus,
transmitting a report based on the examination result to the external apparatus,
inferring, based on the examination result for the patient in a past, examination details and an examination timing of a subsequent examination of the patient,
determining whether or not read-status information indicating that the examination result were read has been received from the external apparatus within a predetermined time period from transmission of the examination result to the external apparatus, and
when it is determined that the read-status information has not been received within the predetermined time period, transmitting a request for generation of an examination order according to the examination details and the examination timing of the subsequent examination of the patient to the external apparatus.

14. The medical assistance method according to claim 13, further comprising:
obtaining a pathology result of a pathological examination of the patient;
estimating acuity information related to acuity based on the pathology result; and
inferring the examination details and the examination timing of the subsequent examination of the patient based on the examination result for the patient in the past and the acuity information.

15. The medical assistance method according to claim 14, further comprising:
determining, based on the acuity information, whether or not the acuity is at a predetermined threshold or more;
when it is determined that the acuity is at the predetermined threshold or more, reserving examination details and an examination time and date according to the request for the generation of the examination order; and
transmitting, to the external apparatus, information indicating that the examination details and the examination time and date according to the request for the generation of the examination order have been reserved, as the request for the generation of the examination order.

16. The medical assistance method according to claim 15, further comprising
when it is determined that the acuity is not at the predetermined threshold or more, transmitting, to the external apparatus, the request for the generation of the examination order according to the examination details and the examination timing of the subsequent examination of the patient.

17. The medical assistance method according to claim 16, further comprising
inferring the examination details and the examination timing of the subsequent examination of the patient based on statistical information based on respective examination results of other patients.
